(19) United States
(12) Patent Application Publication
(10) Pub. No.: US 2010/0212050 A1
(43) Pub. Date: Aug. 19, 2010

(54) PLANTS OVER-EXPRESSING PME

(75) Inventors: Oded Shoseyov, Karmei Yosef (IL); Uzi Kafkafi, Rechovot (IL); Miron Abramson, Karmei Yosef (IL); Ziv Shani, Mazkeret Batia (IL)

Correspondence Address: MARTIN D. MOYNIHAN d/b/a PRTSI, INC., P.O. BOX 16446, ARLINGTON, VA, 22215, US

(73) Assignees: CBD Technologies Ltd., Rechovot (IL); Yissum Research Development Company of the Hebrew University of Jerusalem, Jerusalem (IL)

(21) Appl. No.: 12/593,620

(22) PCT Filed: Mar. 26, 2008
(86) PCT No.: PCT/IL2008/000418

§ 371 Date: Mar. 31, 2010

Related U.S. Application Data
(60) Provisional application No. 60/907,362, filed on Mar. 29, 2007.

(51) Int. Cl. A01H 5/12; A01H 5/00

(52) U.S. Cl. 800/300; 800/295; 800/298

(57) ABSTRACT

The present invention relates to compositions and methods for over-expressing pectin methylesterases (PMEs), particularly type 1 PMEs (PME1s), in plants while maintaining growth as compated to the wild type. The plants of the invention have improved properties compared to the wild type, particularly improved resistance to salinity.

### FIELD OF THE INVENTION

The present invention relates to compositions and methods for over-expressing pectin methylesterases (PMEs), particularly type 1 PMEs (PME1s), in plants while maintaining growth as compated to the wild type. The plants of the invention have improved properties compared to the wild type, particularly improved resistance to salinity.

### BACKGROUND INFORMATION

Soil salinity is an increasing threat for agriculture and is a major factor in reducing plant productivity. Soil salinity is a severe and increasingly limiting constraint on agricultural productivity because it inhibits growth and crop yield. Indeed, over 20% of the irrigated lands in the world have been damaged by salt. Consequently, salinity is a threat to the world food supply. In view of the 40% increase in the human population in less than the past 50 years, crop production must increase if food security is to be ensured.

Sodium is toxic to cell metabolism and has deleterious effect on the functioning of some enzymes. High concentrations of sodium can cause osmotic imbalance, membrane disorganization, reduction in growth and inhibition of cell division and expansion. It also can lead to reduction in photosynthesis and production of reactive oxygen species.

Salinity tolerance has been considered a quantitative trait involving multiple genes, and improvement via traditional plant breeding programs has met with only limited success. Flowers, T. J., J. Exp. Bot., 55:307-319 (2004). The first plant organ to encounter saline medium and potentially the first site of damage under salt stress are the roots. The root cell plasma membrane is an important control point for the exclusion, or selective uptake, of essential ions. Lazof and Bernstein, Adv. Bot. Res., 29:113-189 (1999).

It has been postulated that sensitivity of root elongation to the various alkaline cations correlates with a high surface charge density and promote sorption of divalent cations, such as Ca²⁺, to the root membrane in competition with Na⁺, thereby retaining better membrane integrity in saline media. Kafkafi, U., Root growth under stress: salinity, Plant Roots. The Hidden Half, 375-391 (1991, 1st ed., Marcel Dekker, Inc., New York).

Moreover, salt-tolerant genotypes of melon were found to show less displacement of membrane-associated Ca²⁺ by NaCl salinity than salt-sensitive genotypes of the same species, but membrane vesicles from those cultivars did not differ in their cation exchange capacity (CEC) or affinity for Ca²⁺, Na⁺ or Mg²⁺. Yermiyahu et al., Plant Soil, 191:67-76 (1997). Since root elongation in these cultivars responds to salinity and to Eisenman's series, it has been suggested that the sensitive Ca²⁺—Na⁺ exchange site resides in the cell wall of the elongation zone (Bernstein & Kafkafi, Root growth under stress: salinity, Plant Roots: The Hidden Half, 787-804. (2002, 3rd ed., Marcel Dekker, Inc., New York).

Pectic polysaccharides are a group of polymers that contain 1,4-linked α-D-galacturonic acid (GalA) residues. These polysaccharides are major components of the primary walls of gymnosperms and dicots. O'Neill et al., The pectic polysaccharides of primary cell walls, Methods in Plant Biochemistry, vol. 2: Carbohydrates, (Dey P. M., ed., London: Academic Press, 415-441 (1990)).

Accumulation of Ca²⁺ has been correlated with the CEC of plant roots and it is attributed to the free carboxyl groups of galacturonic acids of cell wall pectins in the middle lamella. Haynes, R. J., Botanical Review, 46:75-99 (1980). Salinity influences the biosynthesis of cell-wall polymers, and wall metabolism. It has been suggested that, in solutions of low ionic strength, plants with higher root CEC compete for divalent cations more effectively, whereas plants with lower root CEC compete for monovalent cations more effectively. Smith and Wallace, Soil Science, 81:97-109 (1956); Asher and Ozanne, Australian Journal of Agricultural Research, 12:755-66 (1961).

Halophytes have been reported to produce large amounts of cell-wall material under saline conditions and to show greater wall plasticity. The increased amount of cell-wall material has been suggested to increase the Ca²⁺-binding capacity of the wall and thus maintain cell growth. Binet, P., Vegetagio, 61:241-46 (1985).

It has been postulated that the salt-tolerant wild barley has a higher pectic polysaccharide content in the cell wall than a salt-sensitive cultivar, where polygalacturonan regions can crosslink with Ca²⁺. Zhong and Läuchli, J. Exp. Bot., 44:773-78 (1993); Suhayda et al., Salinity alters root cell wall properties and trace metal uptake in barley, Biochemistry of Metal Micronutrients in the Rhizosphere (Manthey J. A., Crowley D. E. and Luster D. G. Eds., Boca Raton Lewis Publishers, 325-342 (1994)). It was also demonstrated that adaptation to salt stress in tobacco cells resulted in higher pectin contents. McCann et al., Plant J., 5:773-85 (1994).

The cell wall of a plant is largely made of polysaccharides, including celluloses, hemicelluloses and pectins. Pectins are secreted into the cell wall in highly methylesterified forms. Afterward, they can be modified by pectinases such as pectin methylesterases (PMEs), which are enzymes that can catalyze the demethylesterification of pectins, releasing acidic pectins and methanol. See Micheli, F., Trends in Pant Science, 6:414-19 (2001).

It is known that many plant PME genes encode so-called pre-pro-proteins. The pre-region is required for protein targeting to the endoplasmic reticulum, while only the mature part of the PME, without the pro-region, is extracted to the cell wall. Several functions for the pro-region have been suggested, including targeting of PME to the cell wall, correct folding of PME, and inhibition of PME enzyme activity. Bush et al., Plant Physiol., 138:1334-46 (2005); Micheli, 2001, supra.

According to recent data from the systematic sequencing of the Arabidopsis genome, PME genes can be divided into two classes. Genes in the first class contain only two or three introns and a long pro region, and genes in the other class contain five or six introns and a short or nonexistent pro region, which, if existent, codes an amino acid sequence of 200, 150, 75, 50 or fewer residues. These two classes have been called type I and type II, respectively. Kaul et al., Nature, 408:796-815 (2000).

The type II sequences have a structure close to that of the PMEs identified in phytopathogenic organisms (bacteria, fungi) and are involved in cell wall soaking during plant infection. PME isoforms involved in microsporogenesis and pollen tube growth are type II PMEs related to bacterial PMEs. Micheli, 2001, supra.

It is significant that the action patterns of plant and fungal PMEs are thought to be different. It is generally proposed that plant PMEs remove methyl-esters in a processive blockwise fashion (single chain mechanism), giving rise to long contiguous stretches (blocks) of un-esterified GalA residues in HG domains of pectin. In contrast, the action of fungal PMEs is generally regarded as random (or multiple chain mechanism), resulting in the de-esterification of single GalA residues per enzyme/substrate interaction. Willats et al., Plant J., 18:57-65 (1999). This, it is also generally accepted to divide the PMEs into two groups: “linear de-methylesterifying” (e.g. Type 1) and “random de-methylesterifying” (e.g. Type 2).

When PMEs act randomly on homogalacturonans, the demethylesterification releases protons that promote the action of endopolygalacturonases and contribute to cell wall loosening. When PMEs act linearly on homogalacturonans, PMEs give rise to blocks of free carboxyl groups that could interact with Ca²⁺, so creating a pectate gel (Micheli, 2001).

Several studies have shown that pectin cation binding is altered by PME (Tieman & Handa, 1994; Pilling et al., 2000). While such studies have demonstrated the over-expression of PME in plants, these plants have inferior properties, most notably a marked decrease in growth compared to the wild type. Hasunuma et al., J. Biotechnol., 111:241-51 (2004).

Thus, there is an acute need to modify plants to increase their tolerance to salinity. In addition, there is a need to maximize the positive properties of plants over-expressing PME while minimizing the negative properties. The subject invention addresses these needs and provides additional advantages as well.

### SUMMARY OF THE INVENTION

The present invention provides a plant that includes an over-expressed PME, particularly PME1, where the plant shows less than a 50% decrease in growth compared to the wild type of plant. Growth can be measured, for example, by mass, average leaf size or height.

Plants of the present invention have improved salinity tolerance compared to the wild type. Salinity tolerance can be measured, for example, by the average length of seedling roots compared to the wild type.

The over-expression system of the invention can include a promoter. The promoter can cause over-expression in just one type of plant cell or in many types of plant cells.

The plants of the invention can also have improved: tolerance to heavy metals, herbicides, particularly cationic herbicides, dye intensity, mechanical properties, as well as other physical properties.

### BRIEF DESCRIPTION OF THE DRAWINGS

FIG. 1 shows the schematic representation of plant-compatible pBINPLUS vectors containing the rcpme1 cDNA under various promoters: (a) CaMV 35S promoter (35S); (b) Cell elongation specific promoter; and (c) 4CL-1 promoter. The entire constructs include a terminator sequence (nos) and a neomycine phosphotransferase gene (nptII), which is used as a selection marker in plant transformation. “LB” and “RB” designate, respectively, the left and the right border of the T-DNA flanking the transformed gene constructs.

FIG. 2 shows the comparison of rcpme1 expression levels between the different transgenic tobacco lines and wild-type. Actin was used as an internal control. RT-PCR was used to determine transcript levels for rcpme1 (top) or actin (bottom) in the different transgenic tobacco lines: (a) 35S-rcpme1 clone 1; (b) 35S-rcpme clone 4; (c) cel1-rcpme1 clone 15; (d) 4cl-1-rcpme1 clone 47; and (e) WT plant.

FIG. 3 shows the results of the PME enzymatic activity assay of the transformed plants of the invention. (U=mmole methanol formation min⁻¹; means±SD)

FIG. 4 shows the transgenic tobacco protein extracts on western blot reacting with anti-RCPME1 antibodies. The columns are as follows: (a) 35S-rcpme1 line 1.3; (b) elongation zone of WT plant; (c) Cel1-rcpme1 line 15.1; (d) 4CL-1-rcpme1 line 44.7; and (e) fully expanded leaf of WT plant. The arrows indicate the 36 kDa recombinant RCPME1.

FIG. 5 shows the growth patterns of the wild type and transformed plants (a) in the absence of NaCl and (b) when exposed to 100 mM NaCl.

FIGS. 6a to 6c are photographs of 35S-RCPME1 and WT plants after exposure to NaCl treatment of two-week-old WT and overexpressing line 1.3.6 seedlings in ¼ MS agar plates (a) No NaCl added; (b) 50 mM NaCl added; and (c) 150 mM NaCl added. FIG. 6d shows the root length (values are mean±SD; P<0.05, Tukey-Kramer test).

FIG. 7 shows the immuno-localization of low-methyl-ester pectin (PAM1 epitope) in stem cross sections of WT and 35S-rcpme1 line 1.3.6. Stem sections were taken from the fifth internode. Scale bars=20 μm.

FIG. 8 shows the nucleic acid sequence of the complete ORF of P. sativum rcpme1 cDNA (accession no. AF056493; SEQ ID NO:1).

FIG. 9a shows transgenic (35S-RCPME1) and WT plants after exposure to CdSO₄ treatment. FIG. 9b shows the root length of the transgenic and WT plants in both control and CdSO₄ media.

### DETAILED DESCRIPTION OF THE INVENTION

The present invention centers around the surprising finding that plants over-expressing PME, particularly PME1, can have many beneficial properties and yet maintain a normal growth phenotype. This is surprising both because the literature has taught that over-expressing PME in plants leads to a low growth phenotype, even dwarfism. It is additionally surprising because pectin is considered a glue-type of substance. Therefore, it may be thought that degrading pectin, rather than strengthening it, is needed to promote growth.

More specifically, the present invention provides a plant over-expressing PME, wherein the plant shows less than a 50% decrease in growth compared to the wild type of plant, more preferably less than a 40% decrease, less than a 30% decrease, less than a 20% decrease, less than a 10% decrease, less than a 5% decrease and, most preferably, no decrease compared to the wild type plant at the same growth stage.

Comparing the over-expressed plant to the wild type at the same stage of development, a change in growth can be measured by the mass of the plant, the average leaf size of the plant or the height of the plant. Less than a 50% change, or an even lesser change as disclosed above, in any of these growth factors is within the scope of the present invention.

As disclosed below, plants the present invention have many beneficial properties compared to the wild type of plant. For example, plants of the present invention have increased resistance to salinity. See Example 6. Increased resistance can be measured, for example, by the increased average length of the seedling roots of the over-expressed plant compared to the wild type. The average length can be at least 5%, 10%, 15%, 20%, 25% or longer. The relative change disclosed herein can occur in mild (e.g., about 50 mM NaCl or less) moderate (e.g., about 100 mM NaCl) or high (e.g., about 150 mM NaCl or higher) salt concentrations.

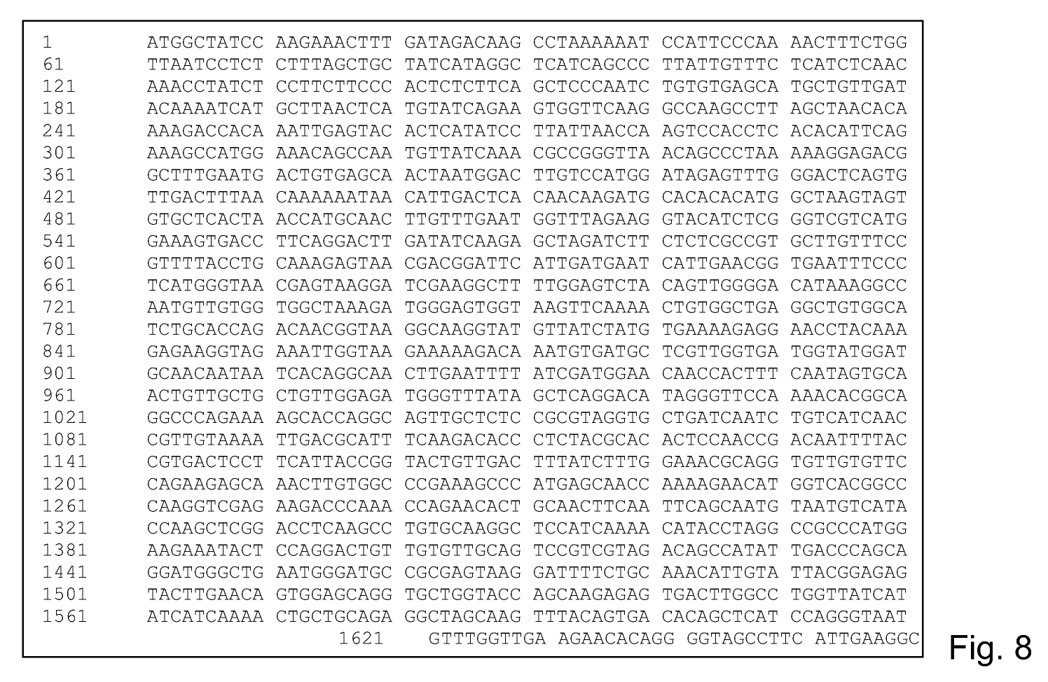

As disclosed herein, PME1 has a longer pro region than PME2. Accordingly, the PME of the present invention includes a pro region that encodes 50, 75, 100, 125, 150 or more amino acids. Preferably, as shown in the examples, the PME is pea rcpme 1 (SEQ ID NO:1; FIG. 8).

Plants of the present invention can over-express PME using a promoter. The promoter can over-express PME in cells during their development or growth after their development or growth or throughout the cell cycle. Similarly, the promoter of the present invention can express PME in only one plant cell type, such as in elongation cells, or in more than one cell type. As shown in the examples below, promoters include CamV 35S, Cel1 and 4CL-1.

The plant of the present invention also has improved tolerance to heavy metals as compared to the wild type using, for example, the growth comparisons disclosed above. The heavy metals include arsenic, barium, cadmium (see Example 8), chromium, cobalt, copper, lead, mercury, nickel, tin, zinc and aluminum. Typical concentrations of these metals are as follows (including a range of +/−20%):

| | Concentration in soil | Concentration in |
|---|---|---|
| Metal | (mg/kg) | groundwater (μg/L) |
| Arsenic | 30 | 30 |
| Barium | 400 | 100 |
| Cadmium | 5 | 2.5 |
| Chromium | 250 | 50 |
| Cobalt | 50 | 50 |
| Copper | 100 | 50 |
| Lead | 150 | 50 |
| Mercury | 2 | 0.5 |
| Nickel | 100 | 50 |
| Tin | 50 | 30 |
| Zinc | 500 | 200 |
| Aluminum | 150 | 80 |

The plants of the present invention also have improved tolerance to herbicides, particularly cationic herbicides, as compared to the wild type using, for example, the growth comparisons disclosed above. Examples of herbicides are paraquat diquat, with typical concentrations of 50 to 100 mg/L.

The plants of the present invention also have improved dye intensity compared to the wild type, which can be measured, for example, by a colorimeter or spectrophotometer.

The plants of the present invention also have improved physical properties compared to the wild type. These physical properties can be measured by Young's modulus, strain at maximum load, energy to break point, water absorbency, swell-ability or toughness. Thus, the present invention provides a paper, textile, yarn, bio-fuel or fiber product with one or more of these properties. These products come from, in whole or in part, a plant of the invention that over-expresses PME.

The plants of the present invention also have an improved mechanical property compared to the wild type, where the mechanical property is measured by tensile strength, resistance to shear, abrasion resistance, frictional coefficient, elasticity or wet strength. Thus, the present invention provides a paper, textile, yarn, bio-fuel or fiber product with one or more of these properties. These products come from, in whole or in part, a plant of the invention that over-expresses PME.

The present invention also includes vectors that include a PME sequence that can cause the over-expression of PME in a plant and yet maintain a growth phenotype that is at least 50%, 60%, 70%, 80%, 90%, 95% or, most preferably, the same or greater as the growth phenotype of the wild type of plant at the same stage of growth.

The present invention also encompasses methods of making the transformed plants of the invention by taking a vector that includes a PME, preferably a PME1, and introducing it into a plant. Preferably, the vector includes a promoter. Preferably, the promoter causes expression of the PME in more than one cell type of the plant and both during and after cell development.

As used herein “PME” are enzymes that can catalyze the demethylesterification of pectins, releasing acidic pectins and methanol. The present invention encompasses all PMEs, which are defined in the literature as disclosed herein and can also be tested for enzymatic activity as disclosed herein. Preferably, the PME of the invention is PME1, as defined herein.

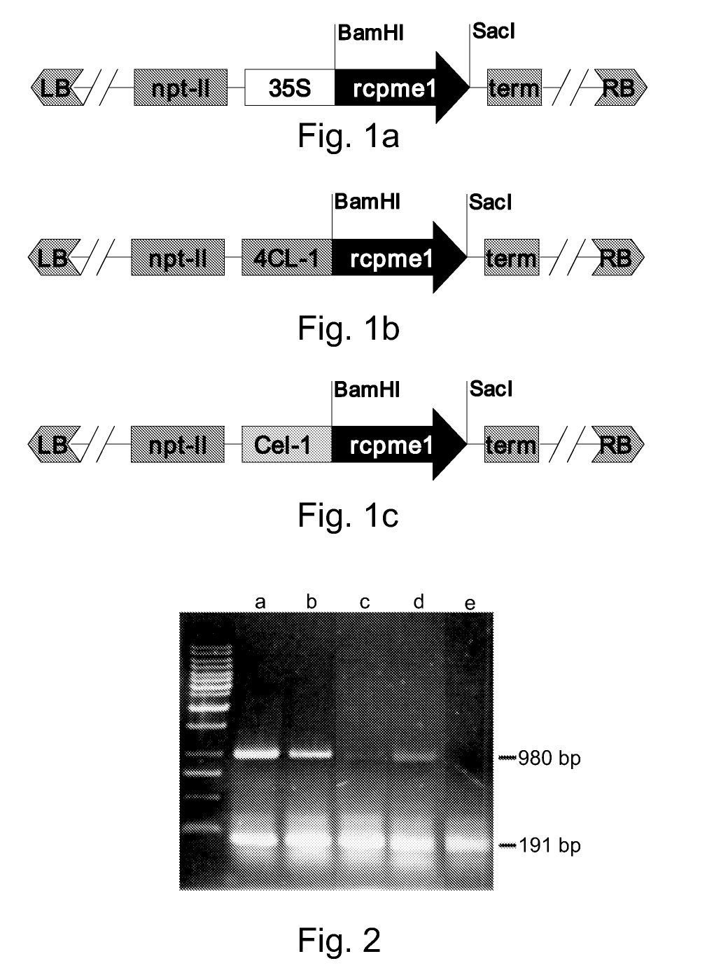

The skilled artisan would know how to acquire and use a PME to carry out the claimed invention. Indeed, the sequence of well over 100 PMEs are known and have been compared and analyzed. Form this analysis, for example, the PME protein has six strictly conserved residues (Gly44, Gly154, Asp157, Gly161, Arg225 and Trp227), as well as six conservative residues (Ile39, Ser86, Ser137, Ile152, Ile159 and Leu223). Markovic and Janecek, Carbohydrate Research, 339:2281-95 (2004) (the listing of PMEs in Table 1 and their sequences listed at www.imb.savba.sk˜janecek/Papers/CE-8/table1.htm, as well as the alignment analysis shown in FIG. 1, is specifically incorporated by reference).

Preferably, the PME of the invention is a plant PME. The skilled artisan would know how to differentiate a plant PME from other PMEs, i.e., those of bacteria and fungi. For example, plant PMEs have the following conserved residues: Ala 187, Gly 224 and Arg278.

The present invention also encompass nucleic acid sequences that have at least 70%, 80%, 90%, 95%, 96%, 97%, 98% and 99% or more homology with SEQ ID NO:1.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res., 12:387-395 (1984).

Another example of algorithm that is suitable for determining percent sequence identity and sequence similarity are the BLAST and BLAST 2.0 algorithms, which are described in Altschul et al., Nuc. Acids Res, 25:3389-3402 (1977) and Altschul et al., J. Mol. Biol., 215:403-410 (1990), respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The gene according to the present invention also includes DNA that hybridizes under stringent conditions to DNA consisting of the nucleotide sequence complementary to DNA consisting of the nucleotide sequence as shown in SEQ ID NO: 1 in the Sequence Listing and that encodes a protein having activity of imparting salt stress tolerance to plants.

The PME according to the present invention also includes DNA that hybridizes under stringent conditions to DNA consisting of the nucleotide sequence complementary to DNA comprising or consisting of SEQ ID NO: 1.

The term “stringent conditions” refers to conditions where what is called a specific hybrid is formed but a non-specific hybrid is not formed. Under such conditions, for example, complementary strands of DNA consisting of a highly homologous nucleic acid, i.e., DNA consisting of a nucleotide sequence exhibiting about 65% or higher, preferably about 75% or higher, more preferably about 85% or higher, and most preferably about 95% or higher, homology to the nucleotide sequence as shown in SEQ ID NO: 1 hybridize, but complementary strands of a nucleic acid having homology lower than the aforementioned level do not hybridize. More specific conditions are constituted by a sodium concentration of 150 mM to 900 mM, and preferably 600 mM to 900 mM, and a temperature of 60° C. to 68° C. and preferably 65° C.

The PME of the present invention can also include on more deletion, addition or substitutions of the encoded protein that would not eliminate its activity, as known by the skilled artisan. The deletion, addition, and substitution of amino acid residues can be carried out by modifying the aforementioned protein-encoding gene via a technique known in the art. Mutation can be introduced to a gene via conventional techniques such as the Kunkel method or the Gapped duplex method, or via a technique in accordance therewith. For example, mutation is introduced using a mutagenesis kit, such as a Mutant-K (Takara) or Mutant-G (Takara) utilizing site-directed mutagenesis or the Takara LA PCR in vitro Mutagenesis series kit (Takara).

Once the nucleotide sequence of the gene according to the present invention is determined, the gene according to the present invention can be then obtained via chemical synthesis, PCR utilizing the cloned cDNA as a template, or hybridization utilizing a DNA fragment having such nucleotide sequence as a probe. Further, modified DNA that encodes the aforementioned gene can be synthesized via, for example, site-directed mutagenesis.

In addition to the meaning described herein, imparting salt stress tolerance to plants can also be evaluated by continuously subjecting the plants to salt stress of 0.3% to 3.0% NaCl for 2 to 8 weeks and then observing the growth conditions thereof in terms of, for example, visual inspection, survival, yield, and amount of growth. The NaCl concentration of salt stress can vary depending on the type of plant. For example, a NaCl concentration can be made to be 0.3% in the case of rice, 1% in the case of barley, and 0.3% to 1% in the case of wheat and maize. When the numerical values obtained in any one, and preferably 2 or more, of the aforementioned terms are higher than those of control plants (e.g., non-transgenic plants), the plants can be evaluated as having activity.

Properties of the products made, in whole or in part, from a transgenic plant of the invention, can be measured, for example, using a universal testing machine (Instron, High Wycombe, UK) Interface type: 1011 series. Sample rate: 10 pts/sec. Crosshead speed: 5 mm/min.

Tensile elastic modulus, or Young's modulus, is an important mechanical property of materials. Young's modulus can be defined as the force required to elongate a material in the elastic regime using relatively small forces that do not irreversibly stretch the material.

The gene or recombinant vector of the present invention can be incorporated in plants by, for example, the Agrobacterium method, the PEG-calcium phosphate method, electroporation, the liposome method, the particle gun method, and microinjection. For example, the Agrobacterium method may employ a protoplast or a tissue section. When a protoplast is employed, the protoplast is cultured together with the Agrobacterium having a Ti plasmid, or it is fused with a spheroplasted Agrobacterium (the spheroplast method). When a tissue section is employed, Agrobacterium is allowed to infect an aseptically cultivated leaf section (a leaf disc) of target plant (the leaf disc method) or a callus (an undifferentiated cultured cell).

Whether or not the gene has been incorporated into the plant can be confirmed via a selectable marker, PCR, Southern hybridization, Northern hybridization, or other means. A selectable marker can be used, for example, the NPTII gene with an antibiotic such as kanamycin. Other examples of selectable markers are well known in the art.

Thus, the recombinant construct of the present invention may include a selectable marker for propagation of the construct. For example, a construct to be propagated in bacteria preferably contains an antibiotic resistance gene, such as one that confers resistance to kanamycin, tetracycline, streptomycin, or chloramphenicol. Suitable vectors for propagating the construct include plasmids, cosmids, bacteriophages or viruses, to name but a few.

In addition, the recombinant constructs may include plant-expressible selectable or screenable marker genes for isolating, identifying or tracking of plant cells transformed by these constructs. Selectable markers include, but are not limited to, genes that confer antibiotic resistances (e.g., resistance to kanamycin or hygromycin) or herbicide resistance (e.g., resistance to sulfonylurea, phosphinothricin, or glyphosate). Screenable markers include, but are not limited to, the genes encoding .beta.-glucuronidase (Jefferson, Plant Molec Biol. Rep, 5:387-405 (1987)), luciferase (Ow et al., Science, 234:856-59 (1986)), and the B and C1 gene products that regulate anthocyanin pigment production (Goff et al., EMBO J, 9:2517-22 (1990)).

In embodiments of the present invention which utilize the Agrobacterium system for transforming plants (see infra), the recombinant DNA constructs additionally comprise at least the right T-DNA border sequence flanking the DNA sequences to be transformed into plant cell. In preferred embodiments, the sequences to be transferred in flanked by the right and left T-DNA border sequences. The proper design and construction of such T-DNA based transformation vectors are well known to those skilled in the art. As another example, DNA can be prepared from a transgenic plant, a DNA-specific primer is designed, and PCR is then carried out. After PCR has been carried out, the amplification product is subjected to agarose gel electrophoresis, polyacrylamide gel electrophoresis, or capillary electrophoresis and stained with ethidium bromide, a SYBR Green solution, or the like, thereby detecting the amplification product as a band. Thus, transformation can be confirmed. Alternatively, the amplification product can be detected via PCR with the use of a primer that has been previously labeled with a fluorescent dye or the like. Further, the amplification product may be bound to a solid phase such as a microplate to thereby confirm the amplification product via, for example, fluorescent or enzyme reactions.

In the present invention, monocotyledonous plants or dicotyledonous plants may be used for transformation. Examples of monocotyledonous plants include those belonging to: Graniineae such as rice, barley, wheat, maize, sugarcane, Zoysia, sorghum, Italian millet, and Japanese millet; Liliaceae such as asparagus, lily, onion, Allium tuberosum, and Japanese dogtooth violet; and Zingiberaceae such as ginger, Zingiber mioga, and Curcuma longa. Examples of dicotyledonous plants include, but are not limited to, those belonging to Brassicaceae such as Arabidopsis thaliana, cabbage, rapeseed, cauliflower, broccoli, and radish; Solanaceae such as tomato, eggplant, potato, and tobacco; Leguminosae such as soybean, garden pea, kidney bean, and alfalfa; Cucurbitaceae such as cucumber, melon, and pumpkin; Umbelliferae such as carrot, celery, and Cryptotaenia japonica; Asteraceae such as lettuce; Malvaceae such as cotton and okra; Chenopodiaceae such as sugar beet and spinach; Myrtaceae such as Eucalyptus and clove; and Salicaceae such as poplar.

In the present invention, examples of plant materials to be transformed include: plant tissues such as a root, stem, leaf, seed, embryo, ovule, ovary, shoot apex (the growing point at the edge of a plant seedling), anther, and pollen; sections of such plant tissues; undifferentiated calluses; and cultured plant cells such as protoplasts prepared by removing cell walls via enzyme processing.

A transgenic plant in the present invention refers to a whole plant, a plant organ (such as a root, stem, leaf, petal, seed, or fruit), a plant tissue (such as the epidermis, phloem, parenchyma tissue, xylem, vascular bundle, palisade tissue, or spongy tissue), or a cultured plant cell.

When a cultured plant cell is to be transformed, an organ or individual may be re-generated from the obtained transformed cell via conventional tissue culture techniques. A person skilled in the art can easily carry out such procedures via a common technique that is known as a method of regenerating a plant from a plant cell. For example, a plant can be regenerated from a plant cell in the following manner.

When plant tissues or protoplasts are used as plant materials to be transformed, they are first cultured in a callus-forming medium that has been sterilized with the addition of, for example, inorganic elements, vitamins, carbon sources, saccharides as energy sources, or plant growth regulators (phytohormones, such as auxin or cytokinin), and indeterminately proliferating dedifferentiated calluses are allowed to form (hereafter, this process is referred to as “callus induction”). The thus formed calluses are transferred to a new medium containing plant growth regulators, such as auxin, and then further proliferated (i.e., subculture).

Callus induction is carried out in a solid medium such as agar, and subculture is carried out in, for example, a liquid medium. This enables both cultures to be carried out efficiently and in large quantities. Subsequently, the calluses proliferated via the aforementioned subculture are cultured under adequate conditions to induce redifferentiation of organs (hereafter referred to as “induction of redifferentiation”), and a complete plant is finally regenerated. Induction of redifferentiation can be carried out by adequately determining the type and quantity of each ingredient in the medium, such as plant growth regulators such as auxin or cytokinin, and carbon sources, light, temperature, and other conditions. Such induction of redifferentiation results in formation of adventitious embryos, adventitious roots, adventitious buds, adventitious shoots, and the like, which leads to growth into complete plants. Alternatively, such items may be stored in a state that pertains before they become complete plants (e.g., encapsulated artificial seeds, dry embryos, or freeze-dried cells and tissues).

The transgenic plant thus obtained acquires tolerance to salt stress. Accordingly, such transgenic plant can be used as a salt stress tolerant plant. The term “salt stress” used herein refers to stress caused by salts that damage physiological functions of plants. For example, salts accumulated in soil lower the water potential of the soil and plants become incapable of absorbing water. Salts include all types of salts that induce growth inhibition, lowered yield, and blight in plants. Examples thereof include sodium salt and magnesium salt.

A salt stress tolerant plant can be produced by breeding a transgenic plant in which the plant's PME gene has been incorporated to an extent such that the resulting transgenic plant can be used as a salt stress tolerant plant. In such a case, a plant may be selected that exhibits tolerance without damage of its physiological functions, growth inhibition, or blight under the aforementioned conditions where salt stress is applied to a plant. The selected plant can be used as a stress tolerant plant at any stage after the tolerant plant has been selected.

The gene of the present invention can be introduced into a plant and then used as a selection marker gene for a transgenic plant. The marker gene of the present invention may be introduced alone or in combination with the other target gene to be expressed.

The marker gene of the present invention may be introduced into a monocotyledonous or dicotyledonous plant. Examples thereof are as listed above, and plants capable of callus formation are preferable.

The marker gene of the present invention can be introduced into, for example; plant tissues such as a root, stem, leaf, seed, embryo, ovule, ovary, shoot apex (the growing point at the edge of a plant seedling), anther, and pollen; sections of such plant tissues; undifferentiated calluses; and cultured plant cells such as protoplasts prepared by removing cell walls via enzyme processing. In the present invention, the marker gene is generally introduced into a tissue section, callus, or protoplast removed from the plant for the purpose of introduction of such gene into the plant, and the introduced marker gene is incorporated in the cell of the plant tissue, and particularly in its chromosome.

When the marker gene is introduced into a plant alone, the marker gene can be ligated to a plasmid to prepare a recombinant vector. When the marker gene is introduced into a plant together with the target gene, however, the marker gene and the target gene are ligated to the same plasmid to prepare a recombinant vector. Alternatively, a recombinant vector that is obtained by ligating the selection marker gene to a plasmid may be prepared separately from a recombinant vector that is obtained by ligating the target gene to a plasmid. When recombinant vectors are separately prepared, both vectors are cotransfected into a host. During vector preparation, a promoter can be ligated to a position upstream of the target gene or the marker gene, and the terminator can be ligated to a position downstream thereof. Examples of promoters include a cauliflower mosaic virus 35S promoter, an actin promoter, and an ubiquitin promoter. An example of a terminator is a nopalin synthase gene terminator. Examples of the methods for introducing the vector into a plant include the aforementioned methods and methods similar thereto.

A gene that exhibits other properties, such as antimicrobial activities against given bacteria, tolerance to a given drug, the capacity for synthesizing a given useful material, sensitivity to a given phytohormone, or morphological properties different from those of the original plant, may be incorporated in the vector together with the marker gene of the present invention to obtain a re-differentiated plant exhibiting such properties.

It is preferable to form a callus from the protoplast or plant tissue into which the marker gene has been introduced in the aforementioned manner and to further culture the formed callus. Methods of callus induction, subculture, and induction of redifferentiation are as described above.

In the present invention, a transgenic plant can be selected by introducing the gene or recombinant vector of the present invention into a plant, culturing the plant in a salt medium (or heavy metal medium or other media as disclosed herein), and selecting the transgenic plant based on the presence or absence of tolerance.

The term “culturing” used herein includes all the culturing processes at each stage of the aforementioned “callus induction,” “induction of redifferentiation,” and “growth into perfect plants (rooting, gemmation, or stem extension).” Whether or not the gene has been incorporated into the plant can be confirmed by culturing the plant in the presence of salt, heavy metal, etc., and inspecting the presence or absence of tolerance in the cultured plant. A plant having such tolerance is selected as the plant into which the gene of interest has been introduced.

In addition to other phenomena disused herein, the term “tolerance” also refers to one or more of the phenomena of callus induction, induction of re-differentiation, or indicia of growth (e.g., rooting, gemmation, or stem extension) normally taking place, without being inhibited by salt or heavy metal, etc.

The selected plant may be allowed to grow in accordance with the aforementioned technique that is commonly adopted in plant tissue culturing. Alternatively, such items may be stored in a state that pertains before they become complete plants (e.g., encapsulated artificial seeds, dry embryos, or freeze-dried cells and tissues).

As discussed above, the cell wall of a plant is largely made of polysaccharides, including celluloses, hemicelluloses and pectins. Pectins are secreted into the cell wall in highly methylesterified forms. Afterward, they can be modified by pectinases or other pectin modifying enzymes such as PMEs. As discussed above, PMEs are enzymes that catalyze the demethylesterification of pectins, releasing acidic pectins and methanol. See Micheli, (2001), supra.

Accordingly, the present invention provides the over-expressed PME proteins be in the cell walls of the plants. Preferably, along with the over-expressed PME is a sequence encoding a signal peptide to allow the PME to enter the cell wall. Such signal peptides are taught, for example, in U.S. Pat. No. 6,184,440.

Calcium ions can reduce the toxicity of sodium to crops grown in saline soils by replacing sodium binding to the cell plasma membrane. Nearly 50% of cellular calcium is bound to the cell wall negatively charged carboxyl groups of de-esterified pectin, which is modified by PMEs. Plant PMEs act linearly on pectin and give rise to blocks of free carboxyl groups that could interact with positive ions such as calcium and sodium.

An aspect of the present invention centers around the idea that increasing pectin negative charge sites by PME will elevate Ca²⁺ binding to the cell wall and plasma membrane and thus promote salt tolerance. As described in the examples below, transgenic tobacco plants were generated expressing pea PME under both constitutive and tissue specific promoters. The transgenic plants displayed a normal phenotype.

Transcription and activity was physiologically and quantitative different according to the various promoters. In all cases, transgenic tobacco cells distribution of de-esterified pectin had increased. As shown in the examples, transgenic root seedlings showed enhanced tolerance to mild salt stress (50-100 mM NaCl) compared to control plants and responded better than the wild type in higher levels of salinity (150 mM NaCl). These results suggest that over-expression of pea PME in tobacco plants increased the total negative charge of the cell wall, acting as an ion-exchange with enhanced calcium binding capacity, thus conferring the plants with salinity tolerance.

As shown in Example 7, a higher ion-exchange capacity for pectin is created in the transgenic plants. Moreover, over-expressing PME can not only work as an ion-exchanger in the Na⁺/Ca²⁺ system, but in other physiological systems that are under electrostatic control as well, thus supporting the idea that plants of the present invention have increased tolerance to heavy metals.

Properties of plant root cell walls appear to influence the efficient use of nutrients as well as tolerance against toxic ions. The ability of root cell walls to retain heavy metals has long been recognized as one of mechanisms that higher plants employ to resist toxic levels of metal ions in contaminated soils (Wang & Evangelou, Handbook of Plant and Crop Physiology (Pessarakli M. Ed., New York: Marcel Dekker, Inc., pp. 695-717 (1995)).

Recently, Hassinen et al. (2006) showed that PME transcript levels were higher in a Zn-tolerant Thlaspi caerulescens plant than in its Zn-sensitive counterpart when exposed to high Zn levels. Elevated expression of PME has been reported in Arabidopsis during long-term Hg exposure (Heidenreich et al., Plant Cell Environ., 24:1227-34 (2001)). Pectin plays an important role in copper accumulation in the fern Lygodium japonicum (Konno et al., J. Exp. Bot., 56:192331 (2005). In confirmation, rcpme1-overexpressing transgenic plants grow more rapidly on 100 μM CdCl₂ than control plants.

The following examples are provided to illustrate but not limit the present invention.

### EXAMPLES

### Experimental Procedures

### Plant Material and Growth Conditions

Pisum sativum cv. Alaska and Nicotiana tabacum-SR1 plants were used. The plants were grown at 26° C. under a 16-h photoperiod, using cool-white fluorescent light (50-60 μE m⁻²s⁻¹), and under greenhouse conditions.

### Example 1

This example describes the procedure for making the vectors containing PME.

PME activity was induced by removing border cells from P. sativum root caps of 25-mm long roots (Stephenson & Hawes, Plant Physiol., 106:739-45 (1994)). After incubation at 24° C. for 2 hours, induced root tips (2 to 3 mm long) were excised, and total RNA was extracted. The complete ORF of P. sativum rcpme1 cDNA (accession no. AF056493; SEQ ID NO:1; FIG. 8) was PCR amplified by using two specific primers.

Primer #1: (BamHI)

(SEQ ID NO: 2)

5′-TTTTGGATCCATGGCTATCCAAGAAACTTTGATAGAC-3′;
and
Primer #2: (SacI)

(SEQ ID NO: 3; restriction enzymes are underlined).

5′-TTTTGAGCTCCTACAGGCCTTCAATGAAGGCTAC-3′

The amplified fragment was subcloned as a BamHI-SacI fragment into pBluescriptII KS+plasmid (Fermentas, Vilnius, Lithuania).

Three constructs were generated in two sequential steps using the binary vector pBINPLUS (Van Engelen et al., Transgenic Res., 4:288-90 (1995)) previously prepared with nos terminator and the nptII gene. The rcpme1 cDNA was cleaved with the BamHI/SacI and ligated into the pBINPLUS which was cleaved with the BamHI/SacI, then the three promoters CaMV 35S, Cel1 and 4CL-1 were ligated into three separated pBINPLUS vectors previously cleaved with the HindIII/BamHI. The constructs were introduced into A. tumefaciens strain LBA4404 for plant transformation.

### Example 2

This example shows the procedure for and results of transforming the tobacco plants.

Leaf-disc transformation was performed with N. tabacum-SR1 plants as described previously (DeBlock et al., Embo J., 3:1681 (1984)). More than 15 independent tobacco transformants were generated for each construct, propagated in vitro and transferred to the greenhouse. The presence of the transgene was confirmed by PCR on genomic DNA using specific primers for rcpme1. T1 seeds obtained by self-pollination of transformants were harvested and selected further on germination medium containing kanamycin (300 mg l⁻¹). The sterilization treatment was for 30 sec in ethanol 70% followed by 5 min NaOCl, 2.5%.

Plants of N. tabacum were transformed with binary vectors carrying the pea rcpme1 coding sequence by using A. tumefaciens mediated transformation. Since the phenotype of transgenic tobacco could not be predicted, three promoters were chosen for different expression patterns. The plant binary vectors contained the CaMV 35S promoter for rcpme1 over-expression, the 4CL-1 promoter for secondary cell wall development and the Cell promoter for expression in elongation zones (FIG. 1).

The transgenic plants were examined for growth alterations. No significant difference was observed between the final heights of the transgenic lines and wild type plants. Furthermore, no difference was found in leaf shape or size.

### Example 3

This example shows the RT-PCR expression analysis of the transformed plants.

Total RNA for RT-PCR was isolated using the Tri-Reagent kit from Sigma (St. Louis, Mo.). First-strand cDNA synthesis was performed using MMLV Reverse Transcriptase (Promega) according to the manufacturer's instructions with 2 μg RNA and oligo-dT primers. cDNA from the first-strand reaction was used as template for PCR. To examine expression of rcpme1, equal amounts of RNA were used for RT-PCR. Two specific primers were used: PMEINS (5′-AACATTGACTCACAACAAGATGCAC-3′; SEQ ID NO:4) and PMEINA (5′-TACGACGGACTGCAACACAAC-3′; SEQ ID NO:5) giving a predicted 980 bp fragment. As a control, PCR amplification of actin was carried out on the same samples using the primers ToAcFor (5′-ATGCCCTCCCACATGCTATTC-3′; SEQ ID NO:6) and ToAcRev (5′-GGTCTTTGACGTCTCGAGTTCCT-3′; SEQ ID NO:7) to give a predicted 191 bp fragment. The number of cycles was optimized and chosen not to exceed the mid-log phase of product yield.

Positive PCR transgenic t₀ plants were characterized. PME expression patterns were examined by RT-PCR. Total RNA was isolated from leaves, stems and elongation zones of tobacco plants. PCR was performed using cDNA from the first-strand reaction with primers specific for the PME clone. Primers specific for tobacco actin were included as an internal control. Differences in PME expression pattern among transgenic plants were found. Expression in Cel1-rcpme1 plants was weak and restricted to the elongation-specific tissues. In the 4CL-1-rcpme1 plants, expression was stronger than in the Cel1-rcpme1 lines but was restricted to the mature tissues. In the 35S-rcpme1 plants, expression was by far the strongest compared to the other transgenic lines and PME could be detected in all tissues (FIG. 2).

### Example 4

This example shows the results of the PME enzymatic assay of transformed plants.

Tobacco plants found positive by PCR, as described above, were then assayed for PME activity by the alcohol oxidase/Purpald procedure (Anthon & Barrett, J. Agric. Food Chem., 52:3749-53 (2004)). Briefly, leaf samples were taken and immediately frozen in liquid nitrogen and ground to a fine powder. Proteins were extracted in extraction buffer (20 mM sodium phosphate, 2 M NaCl, 0.01% Tween 20; pH 7.5), with extracts clarified by centrifugation. The protein concentration of the recovered supernatant was determined by the Bradford method (Bradford, Anal. Biochem., 72:248-254 (1976)) and adjusted to the same value for all samples. The reaction mix included: phosphate buffer pH 7.5, 0.1 U alcohol oxidase (Sigma), 100 μg protein, 150 μg pectin (BDH Chemicals Ltd), 500 μg Purpald (Sigma) in a final volume of 300 μl. Mix was added to an ELISA plate (Nunc); after 30 min at 30° C., and samples were read in a Micro Plate Reader (Bio-Rad) at 550 nm. 1 unit of activity=1 mmole formation of methanol per minute. ε_{550 nm}=22600 M⁻¹ cm⁻¹. Anthon & Barrett, J. Agric. Food Chem., 52:3749-53 (2004).

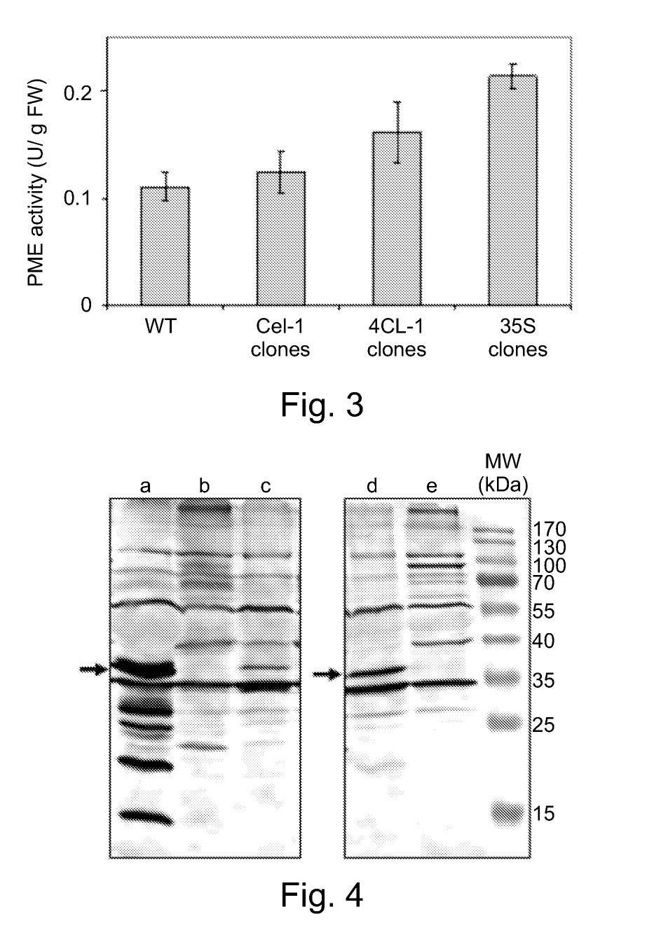

As shown in FIG. 3, PME activity in mature leaves from plants over-expressing 35S-rcpme1 was two-fold higher than in control plants and 4CL-1-rcpme1 plants showed 1.5-fold more activity that in wild type plants.

### Example 5

This example shows the immunological determination of levels of RCPME1 protein in transformed plants.

Antibodies were generated after prediction of the antigenic sites in RCPME1 using the Jameson and Wolf method (GenScript Corp, Piscataway, N.J. USA). Western blot analysis was performed as described in Ausubel et al., Current Protocols in Molecular Biology (John Wiley and Sons, Inc., U.S. (1998)). Crude extracts were prepared for Western blotting as described above. Approximately 100-ng protein samples were subjected to 10% SDS-PAGE. The protein was then transferred to a nitrocellulose membrane (Amersham Biosciences, Piscataway, N.J., USA) using a Mini Trans Blot Cell (Bio-Rad) for 2 hours in cooled transfer buffer with 10% methanol and a steady current of 90 V. After the transfer, the membrane was dipped in a blocking solution for 1 hour at room temperature and overnight at 4° C. The following day, membranes were incubated with anti-PME polyclonal primary antibody (GenScript Corp) for 1 hour, washed three times with TBS-T and incubated once more with an alkaline phosphatase-conjugated secondary antibody. The membrane was then developed with nitro blue tetrazolium and 5-bromo-4-chloro-3-indolyl phosphate-p-toluidine salt as substrates.

Protein analysis correlated well with the activity assay (Example 4) and the RT-PCR (Example 3). The over-expressing line displayed the strongest band at the predicted 36 kDa (FIG. 4). A lower band that appears both in transgenic and wt plants at 34 kDa probably corresponded to the tobacco's internal PME. The 4CL-1-rcpme1 samples were taken from old leaves and cel1-rcpme1 samples were taken from elongation zones.

### Example 6

This example shows the salinity tolerance of the transformed plants.

To assess the relative salinity tolerance of plants, seeds or 3 day-grown seedlings of wild type and the three homozygous T₂ transgenic lines were transferred to ¼ MS medium (Dushefa) supplemented with 50, 100, 150 or 200 mM NaCl to impose salinity stress, or to plain ¼ MS medium as the experimental control. The seedlings were maintained under culture-room conditions, and their root growth was monitored under stress.

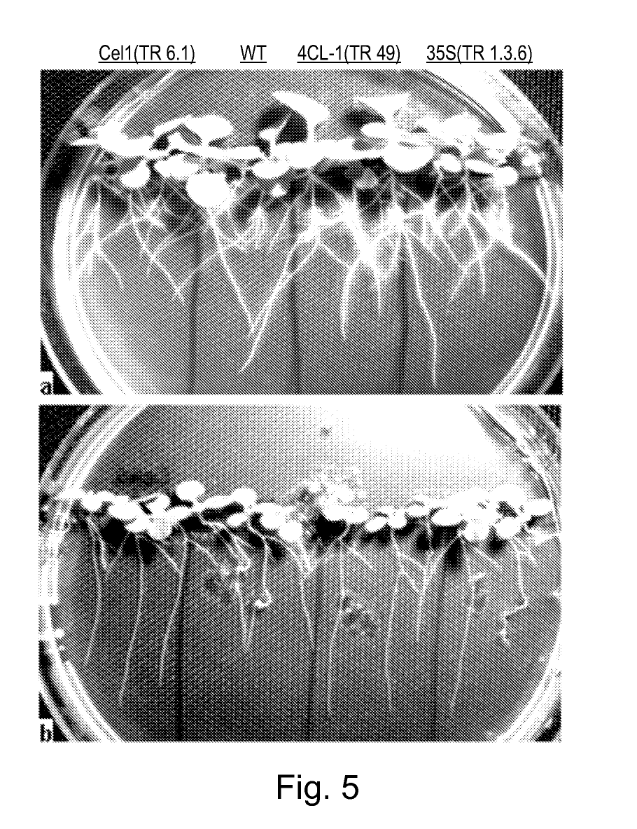

Salt tolerance of T₂-generation transgenic seedlings was checked by transferring the seedlings to various concentrations of NaCl and monitoring their growth for 21 days. All four plant lines-WT, 4CL-1-rcpme1, Cel1-rcpme1, and 35S-rcpme1, showed the same growth pattern and timing in the absence of NaCl (FIG. 5a). When exposed to 100 mM NaCl, the WT plants showed a drastic reduction in growth, whereas the transgenic lines tolerated this degree of salinity (FIG. 5b).

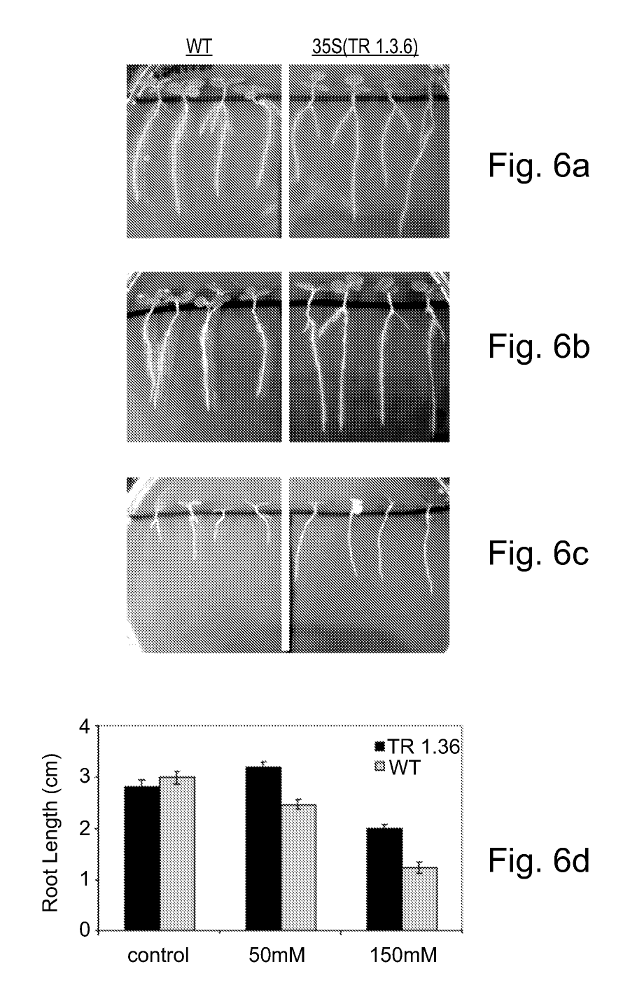

WT and T₂ 35S-rcpme1 seedlings sown on ¼ MS agar plates with or without NaCl addition were then compared. Photographs were taken after 14 days and seedling root length was measured and analyzed by Tukey-Kramer test: P values of <0.05 were considered statistically significant. Significant differences were observed between the 35S-rcpme1 and WT plants after exposure to NaCl treatment (FIG. 6). The elongation of control roots was reduced by about 60% in the presence of 150 mM NaCl, whereas the growth of the transgenic roots was less impaired (29.6% reduction) by this high salinity, indicating increased tolerance to salt stress. Under 50 mM NaCl, elongation in WT plants showed a slight reduction (15%), whereas elongation of transgenic roots showed a slight increase (FIG. 6d).

### Example 7

This example shows the results of immunofluorescence microscopy of the transformed plants, specifically, that their increases tolerance to salinity is related to the density of their negative charge.

To localize pectin, which was de-esterified blockwise, PAM1 antibody (provided by Dr. Paul Knox, Center of Plant Sciences, University of Leeds, UK) was used as described by Willats et al., Plant J., 18:57-65 (1999). Tobacco plants were grown in the greenhouse for 60 days. Regions (0.5 cm long) of stem and petiole were excised and sectioned by hand to a thickness of about 100-300 μm. Sections were immediately placed in fixative consisting of 4% paraformaldehyde in 50 mM PIPES, 5 mM MgSO₄, and 5 mM EGTA. Following 30 minutes of fixation, sections were washed in the PIPES buffer and then incubated for 1 hour in primary antibody diluted in 5% Milk/PBS. PAM1 ScFv antibody was used at a ˜ 1/20 dilution. Sections were washed by gently rocking in PBS prior to incubation for 1 hour in anti-6X His secondary antibody (Sigma). For visualization of PAM1 binding, a tertiary antibody conjugated to FITC (Sigma) was used. After washing in PBS, sections were visualized under a fluorescent microscope (Olympus BX40).

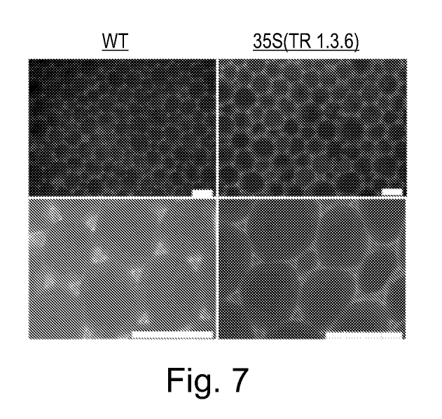

The salinity tolerance of transgenic plants could be related to the density of their negative charge. Since the degree of methylation of pectin defines the extent of the cell wall's negative charge, an attempt was made to determine the degree of methylation of the cell-wall pectin. Homogalacturonan (HG) with different degrees and patterns of methylesterification is recognized by various antibodies (Willats et al., 2000), and the target of the antibody is polygalacturonates dimerized by Ca²⁺. Therefore, a monoclonal antibody (PAM1), which recognizes approximately 20 to 30 contiguous unesterified GalA residues in HG, was used. PAM1 shows up as green fluorescence (FIG. 7). PAM1 antibodies localized pectin in the cell wall of all cells in both WT and transgenic plants as indicated by bright fluorescence. The fluorescence appears brighter in over-expressing lines than in the WT, which shows that the content of low-methyl-ester pectin is higher in 35S-rcpme1 than in the WT. Furthermore, the distribution pattern differed among the lines: in the over-expressing line, the entire intercellular space shows strong fluorescence whereas in the WT, the regions of maximum fluorescence are the cell junctures, indicating distribution of blocks of de-esterified pectin throughout the cells of 35S-rcpme1 plants. Thus, based on the enzymatic mechanism of PME, it modifies the properties of the cell wall as it acts linearly on pectin to form de-methylesterified pectin residues, thus generating blocks of free carboxylic groups or a higher ion-exchange capacity for interaction with divalent cations.

### Example 8

This example shows that the plants showed increase tolerance to cadmium compared to the wild type.

As shown in Example 6, the transgenic plants of the subject invention displayed higher tolerance to NaCl in comparison to WT plants. If this salt tolerance mechanism is not specific to sodium ions, higher tolerance should also be exhibited toward other divalent ions as well.

Cadmium (Cd) is a widespread toxic heavy metal. The uptake of Cd ions is in competition with nutrients, such as potassium, calcium and magnesium. Accordingly and similar to sodium ions, Cd ions significantly reduces growth, both in roots and in stems, and interrupt the normal H⁺/K⁺ exchange and activity of plasma membrane ATPase.

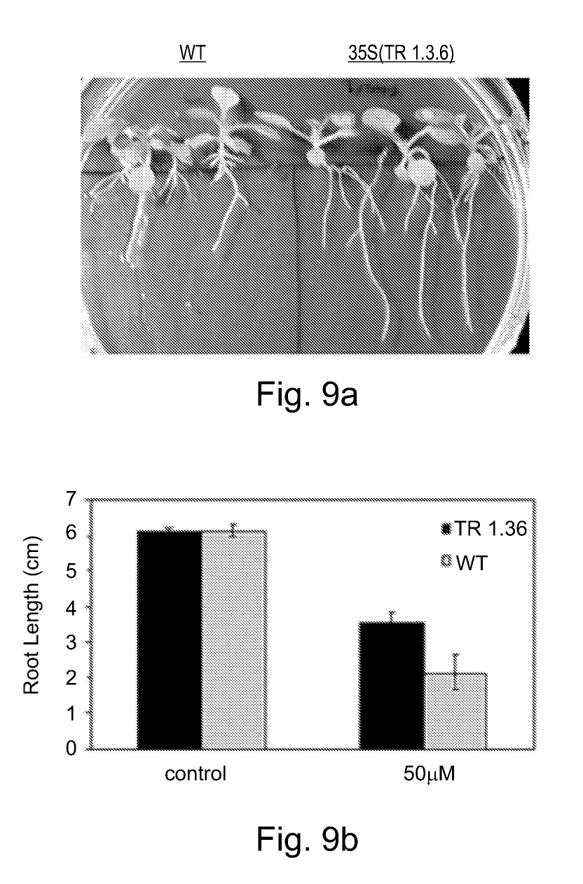

To show that plants expressing rcpme1 are more resistant than WT to Cd²⁺, T2 seeds were germinated and grown for 3 weeks on ½ MS agar plates with or without additional CdSO₄. In medium containing 50 μM CdSO₄ the over expressing transgenic line (TR 1.3.6) grew better than WT plants. FIG. 9A. The elongation of transgenic roots was reduced by 42% while WT roots elongation have decreased by 65%, while in the control medium, the growth of the rcpme1 transgenic tobacco plants was similar to that of the WT plants. FIG. 9b.

All references cited herein are incorporated in their entirety. It is appreciated that the detailed description above is intended only to illustrate certain preferred embodiments of the present invention. It is in no way intended to limit the scope of the invention, as set out in the claims.

1. A plant comprising an over-expressed PME type 1, wherein the plant shows less than a 50% decrease in growth compared to the wild type of plant.

2-4. (canceled)

5. The plant of claim 1, wherein the plant shows less than a 10% decrease in growth compared to the wild type of plant.

6. The plant of claim 1, wherein the plant shows less than a 5% decrease in growth compared to the wild type of plant.

7. The plant of claim 1, wherein the plant shows no decrease in growth compared to the wild type of plant.

8. The plant of claim 1, wherein the decrease in growth is measured by the mass of the plant.

9. The plant of claim 1, wherein the decrease in growth is measured by the average leaf size of the plant.

10. The plant of claim 1, wherein the decrease in growth is measured by the height of the plant.

11. The plant of claim 1, which has improved salinity tolerance as compared to the wild type.

12. The plant of claim 11, wherein the plant is in a solution with a concentration of at least 50 mM NaCl.

13. The plant of claim 12, wherein salinity tolerance is measured by the length of the seedling roots of the over-expressed plant compared to the wild type.

14. The plant of claim 13, wherein the average root of the seedling is at least 5% longer compared to the roots of the wild type.

15-16. (canceled)

17. The plant of claim 1, wherein the PME is a plant PME.

18. The plant of claim 1, wherein the PME has a pro region that encodes 50 or more amino acids.

19. The plant of claim 1, wherein the PME is pea rcpme 1 (SEQ ID NO:1).

20-24. (canceled)

25. The plant of claim 1, which has improved tolerance to heavy metal as compared to the wild type.

26. The plant of claim 25, wherein said heavy metal is selected form the group consisting of arsenic, barium, cadmium, chromium, cobalt, copper, lead, mercury, nickel, tin and zinc.

27-28. (canceled)

29. The plant of claim 1, which has improved tolerance to an herbicide as compared to the wild type.

30-32. (canceled)

33. The plant of claim 1, which has improved dye intensity compared to the wild type as measured by a colorimeter or spectrophotometer.

34. The plant of claim 1, which has an improved physical property compared to the wild type, where the physical property is measured by Young's modulus, strain at maximum load, energy to break point, water absorbency, swell-ability or toughness.

35. (canceled)

36. The plant of claim 1, where the PME is over-expressed in the cell wall of the plant.

37. (canceled)


SEQUENCE LISTING (7 sequences)

SEQ ID NO: 1 - DNA, 1659 bp, Pisum sativum
atggctatccaagaaactttgatagacaagcctaaaaaatccattcccaaaactttctggttaatcctctctttagctgctatcataggctcatcagcccttattgtttctcatctcaacaaacctatctccttcttcccactctcttcagctcccaatctgtgtgagcatgctgttgatacaaaatcatgcttaactcatgtatcagaagtggttcaaggccaagccttagctaacacaaaagaccacaaattgagtacactcatatccttattaaccaagtccacctcacacattcagaaagccatggaaacagccaatgttatcaaacgccgggttaacagccctaaaaaggagacggctttgaatgactgtgagcaactaatggacttgtccatggatagagtttgggactcagtgttgactttaacaaaaaataacattgactcacaacaagatgcacacacatggctaagtagtgtgctcactaaccatgcaacttgtttgaatggtttagaaggtacatctcgggtcgtcatggaaagtgaccttcaggacttgatatcaagagctagatcttctctcgccgtgcttgtttccgttttacctgcaaagagtaacgacggattcattgatgaatcattgaacggtgaatttccctcatgggtaacgagtaaggatcgaaggcttttggagtctacagttggggacataaaggccaatgttgtggtggctaaagatgggagtggtaagttcaaaactgtggctgaggctgtggcatctgcaccagacaacggtaaggcaaggtatgttatctatgtgaaaagaggaacctacaaagagaaggtagaaattggtaagaaaaagacaaatgtgatgctcgttggtgatggtatggatgcaacaataatcacaggcaacttgaattttatcgatggaacaaccactttcaatagtgcaactgttgctgctgttggagatgggtttatagctcaggacatagggttccaaaacacggcaggcccagaaaagcaccaggcagttgctctccgcgtaggtgctgatcaatctgtcatcaaccgttgtaaaattgacgcatttcaagacaccctctacgcacactccaaccgacaattttaccgtgactccttcattaccggtactgttgactttatctttggaaacgcaggtgttgtgttccagaagagcaaacttgtggcccgaaagcccatgagcaaccaaaagaacatggtcacggcccaaggtcgagaagacccaaaccagaacactgcaacttcaattcagcaatgtaatgtcataccaagctcggacctcaagcctgtgcaaggctccatcaaaacatacctaggccgcccatggaagaaatactccaggactgttgtgttgcagtccgtcgtagacagccatattgacccagcaggatgggctgaatgggatgccgcgagtaaggattttctgcaaacattgtattacggagagtacttgaacagtggagcaggtgctggtaccagcaagagagtgacttggcctggttatcatatcatcaaaactgctgcagaggctagcaagtttacagtgacacagctcatccagggtaatgtttggttgaagaacacaggggtagccttcattgaaggc

SEQ ID NO: 2 - DNA, 37 bp, Artificial sequence
  Single strand DNA oligonucleotide
ttttggatccatggctatccaagaaactttgatagac

SEQ ID NO: 3 - DNA, 34 bp, Artificial sequence
  Single strand DNA oligonucleotide
ttttgagctcctacaggccttcaatgaaggctac

SEQ ID NO: 4 - DNA, 25 bp, Artificial sequence
  Single strand DNA oligonucleotide
aacattgactcacaacaagatgcac

SEQ ID NO: 5 - DNA, 21 bp, Artificial sequence
  Single strand DNA oligonucleotide
tacgacggactgcaacacaac

SEQ ID NO: 6 - DNA, 21 bp, Artificial sequence
  Single strand DNA oligonucleotide
atgccctcccacatgctattc

SEQ ID NO: 7 - DNA, 23 bp, Artificial sequence
  Single strand DNA oligonucleotide
ggtctttgacgtctcgagttcct